(19) United States
(12) Patent Application Publication
(10) Pub. No.: US 2003/0127506 A1
(43) Pub. Date: Jul. 10, 2003

(54) Decontaminating mailbox

(76) Inventor: Fritz Braun JR., Satellite Beach, FL (US)

Correspondence Address: BEUSSE, BROWNLEE, BOWDOIN & WOLTER, P. A., 390 NORTH ORANGE AVENUE, SUITE 2500, ORLANDO, FL, 32801, US

(21) Appl. No.: 10/310,278

(22) Filed: Dec. 5, 2002

Related U.S. Application Data
(60) Provisional application No. 60/336,823, filed on Dec. 5, 2001.

(51) Int. Cl. B65D 91/00

(52) U.S. Cl. 232/31

(57) ABSTRACT

A mailbox for decontaminating a mail parcel and the contents of the parcel. The mailbox comprises ultra-violet lamps operative to produce UV-C radiation and collateral ozone. The radiation destroys pathogens on the surface of the parcel and air circulating within the mailbox, as for instance driven by a fan in the mailbox, allows the ozone to penetrate the parcel envelope to contact the contents and destroy pathogens on the contents.

This patent application claims the benefit of Provisional Patent Application No. 60/336,823 filed on Dec. 5, 2001.

### FIELD OF THE INVENTION

The present invention relates generally to decontamination to kill pathogens, and more specifically to the decontamination of mail by a decontaminating mailbox.

### BACKGROUND OF THE INVENTION

The recent anthrax attacks through the contamination of mail have been well-publicized in the press. The attacks have led to several deaths and many near-fatalities, and although certain individuals were targeted, others were exposed to the deadly bacteria through cross-contamination. The perpetrator has not been determined and the threat of future attacks remains high. Even if the number of targets is small, cross-contaminated mail can affect literally millions of people, with no warning until after exposure. The use of anthrax to spread death and destruction is especially pernicious as the perpetrator leaves behind little evidence and there is no advance warning.

Ultraviolet light, a form of electromagnetic radiation in the near-visible portion of the spectrum, is classified in three wavelength ranges: UV-C, ranging from about from 100 nm (nanometers) to about 280 nm; UV-B, ranging from about 280 nm to about 320 nm; and UV-A, ranging from about 320 nm to about 400 nm. Each wavelength range has specific uses and effects. UV-A generates photochemical reactions, UV-B is more rapidly absorbed than UV-A due to its higher energy and generates erythmal reactions, UV-C generates germicidal reactions and ozone. A suntan is a reaction to UV-A, which is a component of sunlight and due its relatively long wavelength can penetrate the atmosphere. Tanning bed lights generate UV-A radiation. It has been found to cause skin disease with extreme exposure. The middle wavelength UV-B radiation is artificially generated to treat skin diseases. UV-C is short-wave ultraviolet radiation, useful for destroying bacteria and other microorganisms in liquids, air, and on surfaces. It is known that germicidal lamps can destroy any microorganism that comes in contact with its powerful UV-C rays. The destruction of viruses and bacteria by germicidal ultraviolet light is accomplished quickly and effectively. The UV-C rays strike the various microorganisms, whether bacteria, virus, yeast, mold or algae, and break through the thin outer membrane. The radiation reaches the organism's DNA, where it causes abrupt modifications. The modified DNA then transmits incorrect codes or messages during cell replication, and this impairment causes the destruction of the microorganism.

The oxygen molecule comprises two oxygen atoms; the ozone molecule comprises three oxygen molecules. Oxygen molecules are broken into atoms by the corona discharge during lightning storms or by ultra-violet light. However, single oxygen atoms cannot exist alone, and typically regroup back into di-atomic oxygen molecules (O2). During this recombination stage some atoms reform into loosely bound tri-atomic oxygen (O3) molecules, which are referred to as ozone in concentrations of more than about 50 parts/million. Due to the loose bond in the O3 oxygen molecule, the molecule is a strong oxidant and an ideal chemical-free purification and disinfecting agent.

There are two conventional techniques for producing O3 molecules. When ultra-violet rays collide with a contaminant such as carbon monoxide (CO) or nitrogen oxides (N02 and N2O) in the presence of oxygen (O2), an O3 molecule is produced. A high voltage discharge (corona) also produces O3. For example, lightning discharges produce ozone, creating the clean fresh smell after a thunderstorm. The voltage of the discharge breaks the O2 oxygen molecules, which tend to reform in groups of three, that is O3. The atoms return to their O2 stable state when one of the oxygen atoms attaches to a contaminant, leaving an O2 molecule behind and forming carbon dioxide and hydrogen. In the event there are no environmental contaminants, the O3 molecule will reform back to O2 within about 20 to 30 minutes at room temperature. Note that the O3 molecule is suicidal, that is, the molecule searches for a contaminant to attack, thus destroying itself. Although harmless to humans, the O3 is lethal to almost all viruses, bacteria, fungi, and cancer cells. The O3 molecule is also known to eliminate pollens, carbon monoxide, chemical gases, benzene, dust mites, mold, mildew and cigarette smoke.

The process of disinfection by tri-atomic oxygen (O3) molecules occurs through the rupture of the cell wall, a more efficient method than even chlorine disinfection, which depends upon diffusion into the cell protoplasm and inactivation of the enzymes. An ozone level of about 0.4 ppm with a four minute exposure has been shown to kill any bacteria, virus, mold and fungus. (1 part per million is equivalent to: 8.345 pounds per million gallons (US)). At higher levels the sanitizing effects are substantially increased. For complete disinfection a surplus or residual O3 should be maintained in the solution to assure that every living microorganism has been contacted and destroyed.

Although there has yet to be discovered an antibiotic that is truly effective against viruses, ozone inactivates viruses on contact, even at very low residual concentrations. In the case of polio, only 0.012 ppm of ozone destroys all viral cells in less than 10 seconds. Mold and mildew are easily controlled by ozone present in air and in water. Giardia and Cryptosporidium cysts are susceptible to ozone, but not affected by normal levels of chlorine when used in a disinfection process.

The antipathogenic effects of ozone have been substantiated for several decades. Its killing action on bacteria, viruses, fungi, and in many species of protozoa, serve as the basis for its increasing use in disinfecting municipal water supplies.

Bacteria are microscopically small single-cell creatures having a primitive structure. They live in foodstuffs and release metabolic products, multiplying by division. The bacteria body is sealed by a relatively solid cell membrane. Their vital processes are controlled by a complex enzymatic system. Ozone interferes with the metabolism of bacterium cells, most likely through inhibiting and blocking the operation of the enzymatic control system. When a sufficient quantity of ozone breaks through the cell membrane, the bacteria is effectively destroyed.

Indicator bacteria in effluents, namely colifonmas and pathogens such as Salmonella, show marked sensitivity to ozone inactivation. Other bacterial organisms susceptible to ozone's disinfecting properties include Streptococci, Shigella, legionella pneumophila, Pseudomonas aerunginosa, Yersinia enterocolitica, Campylobacterjejuni, Mycobacteria, Kelbsiella pneumonia, and Eschenichia coli. Ozone destroys both aerobic and importantly, anaerobic bacteria, which are mostly responsible for the devastating sequel of complicated infections, as exemplified by decubitus ulcers and gangrene.

Viruses are small, independent particles, built of crystals and macromolecules. Unlike bacteria, they multiply only within the host cell. Ozone destroys viruses by diffusing through the protein coat into the nucleic acid core, resulting in damage to the viral RNA. At higher concentrations, ozone destroys the capsid or exterior protein shell by oxidation. Numerous families of viruses including poliovirus I and 2, human rotaviruses, Norwalk virus, Parvoviruses, and Hepatitis A, B and non-A non-BC, among many others, are susceptible to the virucidal actions of ozone. Most research efforts on ozone's virucidal effects have centered upon ozone's propensity to break apart lipid molecules at sites of multiple bond configuration. Indeed, once the lipid envelope of the virus is fragmented, its DNA or RNA core cannot survive. Ozone's effects on non-enveloped viruses are also known.

Fungi families inhibited and destroyed by exposure to ozone include Candida, Aspergilus, Histoplasma, Actinomycoses, and Cryptococcus. The walls of fungi are multilayered and composed of approximately 80% carbohydrates and 10% of proteins and glycoproteins. The presence of many disulfide bonds had been noted, making this a possible site for oxidative inactivation by ozone.

The typical dosage and reaction time for ozone to destroy a particular pathogen have been studied and are know to those skilled in the art. See for example, the following references: “Bactericidal Effects of High Airborne Ozone Concentrations On Escherichia Coli and Staphylococcus Aureus”, Ozone Science and Engineering, Vol. 20, 1998; “The Use of Ultraviolet Light for Microbial Control”, Ultrapure Water, April 1989; William V. Collentro, “Treatment of Water with Ultraviolet Light—Part 1”, Ultrapure Water, July/August 1986; W. J. Wlford, J. V. D. Eude, “An Investigation of the Merits of Ozone as an Aerial Disinfectant”, J Hyg., 42:240-265 (1942); T. H. Heindel, R. Streib, K. Botzenhart, “Effect of Ozone on Airborne Microorganisms”, Zbl. Hygiene 194:464-480 (1993).

### BRIEF SUMMARY OF THE INVENTION

The present invention is an enclosure (a mailbox, for example) for decontaminating an item placed therein. The decontaminating apparatus includes an ultra-violet energy source disposed within the enclosure for destroying pathogens on the surface of the item. Ozone generated by an ozone-producing source, also disposed within the enclosure, penetrates through the porous surface layer of the item to decontaminate the interior contents. The ozone is driven through the pores by establishing an air flow or pressure differential in the enclosure. In the event several items are stacked within the enclosure, the ozone can penetrate between the individual items to decontaminate the contacting surfaces of adjacent items. The invention is targeted at the spore-type anthrax bacteria, although the ultra-violet energy and the ozone will destroy many different types of pathogens, including bacterial, viruses and fungi.

### BRIEF DESCRIPTION OF THE DRAWINGS

The foregoing and other features of the invention will be apparent from the following more particular description of the invention, as illustrated in the accompanying drawings, in which like reference characters refer to the same parts throughout the different figures. The figures are not necessarily to scale, emphasis instead being placed upon illustrating the principles of the invention.

FIG. 1 is a cut-away view of a mailbox constructed according to the teachings of the present invention;

FIG. 2 is an end view of the mailbox;

FIG. 3 is a schematic representation of the various monitoring and controlling elements of the mailbox constructed according to the teachings of the present invention.

### DETAILED DESCRIPTION OF THE INVENTION

Before describing in detail the particular decontaminating mailbox in accordance with the present invention, it should be observed that the present invention resides primarily in a novel combination of hardware elements and software steps. Accordingly, the elements have been represented by conventional elements in the drawings, showing only those specific details that are pertinent to the present invention, so as not to obscure the disclosure with structural details that will be readily apparent to those skilled in the art having the benefit of the description herein.

A mailbox constructed according to the teachings of the present invention reduces potential biological threats perpetrated through the mails by the enclosure of anthrax bacteria within a mail parcel. The invention significantly reduces the risk of contacting certain pathogens by handling mail or exposing oneself to mail-borne or air-borne pathogens on the mail envelope or within the envelope. Also, by killing the pathogens in the mailbox, the likelihood of carrying the contamination into the user's home or business establishment is reduced. Generally, contamination by the anthrax bacteria (whether enclosed in the mail parcel directly or by contact of the parcel with a directly contaminated parcel) is eliminated through the use of ultraviolet light and the collateral production of ozone gas to decontaminate the contents of a mailbox. In one embodiment, the decontamination process is executed utilizing two simultaneous processes: a) the controlled irradiation provided by UV-C light for surface decontamination of the mailbox interior and surface decontamination of the mail or parcels placed within the mailbox, and b) decontamination of the contents of the mail or parcels by ozone penetration through the envelope into the contents of the mail or parcel. The ozone molecules are hundreds of times smaller than the voids within a sheet of paper or an envelope. The penetrating ozone gas is produced as a collateral effect of the UV-C radiation.

Although the teachings of the present invention are described in conjunction with a pole-mounted street-accessible mailbox (known as a standard type T1 US mailbox), for use by a homeowner or small business owner, to remove contamination present on and/or within the mail, the invention is not so limited. The teachings are applicable to any enclosure where mail, parcels or other items that may have been directly or indirectly contaminated are located. For example, routine decontamination of all mail can be performed by placing mail within an enclosure constructed according to the present invention. The apparatus and method for removing the contamination according to the teachings of the present invention destroys not only contamination on one or more surfaces of the item, but also contamination that may be present within the item, such as for instance on paper within an envelope. The present invention also destroys pathogens on contacting surfaces of adjacent stacked items, such as envelopes stacked within a mailbox.

In one embodiment, the mailbox comprises three ultraviolet lamps that emit about 40,000 microwatts-second/cm² of ultra-violet energy, and advantageously also form ozone at production rate of approximately 2.4 grams/hr. One example of suitable lamps include type GSL238T5L/C UV available from Atlantic Ultraviolet Corporation of Hauppauge, N.Y. These lamps include a quart envelope that maximizes the production of UV-C radiation. The spectral output of the lamps has a peak at a wavelength of about 254 nm (within the UV-C range) and produces collateral ozone. This combination of UV-C radiation and ozone is sufficient to kill virtually any virus, bacteria or other pathogen residing on an exposed surface of an item in the mailbox, on adjacent surfaces between two items and within an item, such as a mail envelope. Three 10 watt lamps used in one embodiment produce the same germicidal irradiation as used in many hospital operating rooms to kill pathogens, such as viruses and bacteria. The collateral ozone production coupled with the creation of a differential pressure within the mailbox to set up an ozone flow (in one embodiment by use of a fan), and taking into account the relatively small volume of the mailbox, allows the ozone to penetrate into the interior of an item having a non-air-restricting surface or cover (i.e., having a porous surface layer or enclosure). It has been determined that the size of an ozone molecule is much smaller than the microscopic openings in the material or paper from which a standard mailing envelope is formed.

Ultraviolet technology is a non-chemical disinfection approach. Thus there is no requirement to mix or add liquid chemicals to effect the decontamination process, making the process according to the present invention simple, inexpensive and low maintenance. UV-C light is germicidal, i.e., it deactivates the DNA of bacteria, viruses and other pathogens, destroying their ability to multiply and cause disease. Specifically, UV-C light causes damage to the nucleic acid of microorganisms by forming covalent bonds between certain adjacent bases in the DNA. The formation of such bonds prevents the DNA from being unzipped for replication, and the organism is thus unable to reproduce. In fact, when the organism tries to replicate, it dies. Generally, the germicidal effect is a function of the combination of energy intensity and exposure time.

Although the present invention is described in the context of killing anthrax bacteria in spore form (other forms are more easily destroyed), it is also effective against viruses and other bacteria, including, for example, infectious hepatitis, influenza, Bacillus Anthracis (anthrax), tuberculosis, Proteus Vulgaris, typhoid fever, cholera, various forms of Streptococcus and Staphylococcus, Legionnaire's Disease, diphtheria, and others.

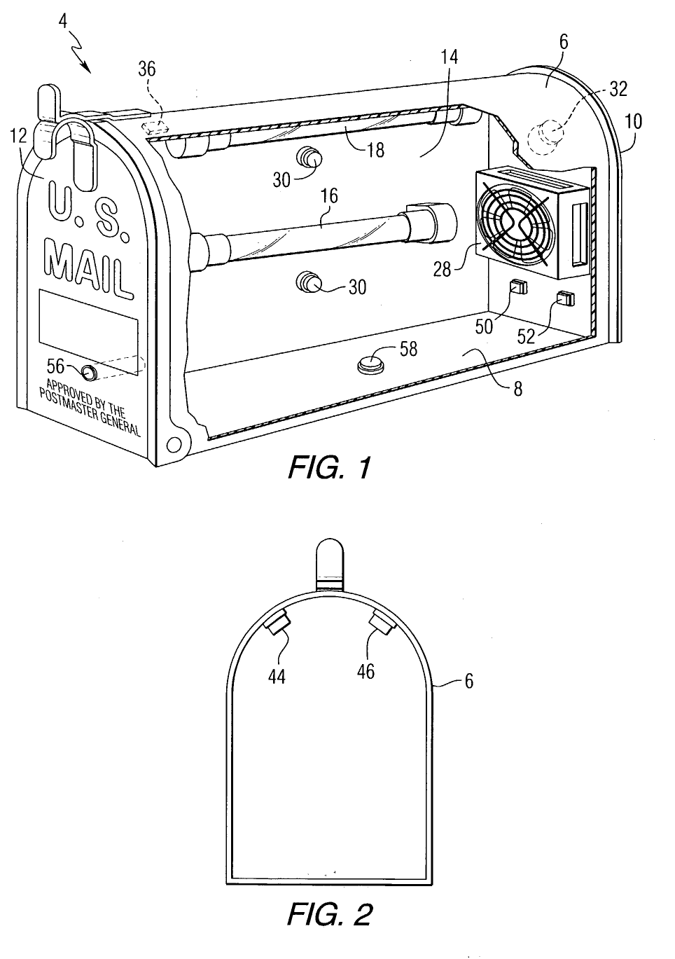

With reference to the cut-away view of FIG. 1, a decontaminating mailbox 4 is formed from a top surface 6, a floor 8, a rear wall 10 and a door 12, all of which cooperate to define an interior chamber 14. In one embodiment, 10-watt ozone producing UV-C lamps 16, 18 and 20 (only two of which are shown in FIG. 1) are disposed within the chamber 14 by affixing to the top surface 6. The lamps are rated for 10,000 hours of operating time at peak ultra-violet emission. Exemplary lamps include the type GSL238T5L/C UV lamps referred to above. Although three lamps are referred to in the FIG. 1 embodiment, the invention does not necessarily require three lamps as the light intensity developed within the mailbox is the operative parameter. Therefore in other embodiments more or fewer than three lamps can be used, as determined by the rated output of each lamp. As shown in this embodiment, the lamps 16, 18 and 20 are equidistantly spaced within the chamber 14 to ensure even irradiation of exposed surfaces of mail and parcels placed within the mailbox 4.

The chamber 14 further comprises a fan 28 internally disposed as shown and affixed to the rear wall 10 for circulating air within the chamber 14. In one embodiment the fan is rated at about 10 cubic feet per minute. In other embodiments differently sized fans or air blowers can be used. It has been determined that providing air circulation within the mailbox advantageously promotes circulation of the ozone and thus the pathogen destruction. In essence, any device that establishes a pressure differential within the mailbox, creating an attendant air circulation allows the ozone to infiltrate porous surfaces within the mailbox 4. Thus the contents of envelopes can be decontaminated as well as the contacting surfaces between adjacent envelopes in the mailbox.

The chamber 14 further comprises one or more UV sensors 30 (two are shown in FIG. 1) positioned proximate the lamps 16, 18 and 20, to monitor the ultra-violet output and provide representative signals thereof to a controller 40, not shown in FIG. 1. In particular, it is desirable to determine when the lamps 16, 18 and 20 have reached about 50% (in one embodiment) of their peak output at the UV-C frequency of about 254 nm. In response to the signal representing the ultra-violet output, the controller 40 controls the on-time of the lamps 16, 18 and 20 to ensure that sufficient UV radiation at the correct frequency has been produced to decontaminate the mailbox contents. In one embodiment the number of UV sensors equals the number of UV lamps.

A heat lamp 32 is also disposed in the chamber 14 to maintain the chamber temperature at the optimum lamp envelope temperature for maximum UV emission from the lamps 16, 18 and 20. The heat lamp 32 is controlled by the controller 40 as described below. Although shown as positioned near the rear wall in FIG. 1, this is not necessarily a required location. Any location within the chamber 14 where the heat produced by the heat lamp 32 is distributed generally throughout the chamber 14 is satisfactory.

A temperature sensor 36, disposed within the chamber 14, provides a temperature signal to the controller 40 for controlling the heat lamp 32 to maintain a predetermined temperature within the chamber 14. In another embodiment, the mailbox 4 comprises more than one temperature sensor 36 to determine the temperature of various regions of the chamber 14 and further to determine whether any temperature gradients exist within the chamber 14.

In one embodiment, two failsafe switches 44 and 46 are mechanically and/or magnetically operable to determine the status of the front door, i.e., opened or closed. See FIG. 2, illustrating an outline end-view of the mailbox 4 with the door 12 removed, and exemplary locations for the failsafe switches 44 and 46. The failsafe switches 44 and 46 are electrically connected to the controller 40 such that control functions can be executed in response to the position of the switches 44 and 46. In the preferred embodiment, the switches 44 and 46 are connected in series, such that both must be closed to allow the controller 40 to apply power to the lamps 16, 18 and 20. If either or both of the switches 44 or 46 are opened while the lamps 16, 18 and 20 are energized, the controller 40 de-energizes the lamps 16, 18 and 20.

In one embodiment the switch 44 is mechanically operated by a force exerted by the closed door against a moveable switch contact of the switch 44. The switch 46 comprises a magnetic reed switch operable in response to the placement of a magnet proximate the switch, causing closure (or opening in another embodiment) of the switch contacts. The magnet, not shown in FIG. 2, is affixed to the door 12 for cooperating with the magnetic reed switch 46, closing (or opening) the switch contacts when the door 12 is closed.

Externally visible lamps or light emitting diodes (LED's) 50 and 52 indicate the current state (i.e., ready, decontaminating) and failures associated with the operation of the mailbox 4. For example, one of the lamps 50 and 52 can indicate a failure condition such as, one or more of the lamps 16, 18 and 20 are not functional, or the heat lamp 32 is not functional.

In one embodiment, a light pipe 56 is mounted within the front door 12 as shown to allow safe visualization of the UV lamp activity. Typically, the light pipe is a passive element for providing a direct visual indication to the user when the lamps 16, 18 and 20 are operating.

A sensor 58 is mounted within the floor 8 for providing a signal to the controller 40 indicative of whether mail or parcels are in the mailbox 4. The sensor 58 can comprise a micro-switch that is depressed in response to the weight of mail or a parcel placed on the floor 8. Alternatively, the sensor 58 comprises a conventional photo-electric device the status of which is determined by the light collected. Thus mail or parcels on the floor 8 block light from entering the sensor 58, a condition which is sensed by the controller 40. Additional sensors, similar to the sensor 58, can be strategically placed within the mailbox 4 (in the door 12 and rear wall 10, for example) to provide a qualitative measure of the amount of material placed within the mailbox 4. In one embodiment this information is used by the controller 40 to control the decontamination duration.

Generally, the mailbox 4 is operative in three modes: a start-up cycle, a decontamination cycle and a post-decontamination or self-cleaning cycle.

The start-up cycle is initiated by opening the door 12 of the mailbox 4, in response to which the controller 40 senses an interrupt based on a change in the position of the switches 44 and 46. The controller 40 waits for door closure, again in response to a change in the position of the switches 44 and 46. The switches 44 and 46 can be implanted as either normally-open or normally-closed switches, with attendant modifications to the controller 40. Herein for the purpose of explaining operation of the mailbox 4, the switches 44 and 46 are assumed to be closed when the door 12 is closed. The controller 40 also monitors the condition of the sensor 58 to determine which of the operating cycles is to be executed.

The decontamination cycle begins when the controller 40 senses, via the switches 44 and 46, that the door 12 is closed, concurrently with the state of the sensor 58 indicating that mail is within the mailbox. 4. The controller 40 monitors the internal temperature of the mailbox 4 to ensure that the internal temperature is at least about 44° C., the optimum ambient temperature for operation of the lamps 16, 18 and 20. Should the temperature be lower than 44° C. (as during cold weather), the controller 40 energizes the heat lamp 32, which produces some ultra-violet radiation during the heating process. To limit temperature gradients within the chamber 14, the fan 28 is energized by the controller 40. In one embodiment the fan 28 is energized whenever the heat lamp 28 is energized.

When the internal temperature reaches about 44° C., as measured by the temperature sensor 36, the controller 40 activates lamps 16, 18 and 20. During the decontamination or UV production interval, the controller 40 monitors the internal temperature of the mailbox 4, energizing the heat lamp 28 as required to maintain the internal temperature at about 44° C. The controller 40 also monitors the individual UV-C output of each of the lamps 16, 18 and 20 in response to signals produced by the ultra-violet sensors 30. Finally the controller 40 monitors the state of the failsafe switches 44 and 46 (providing an indication of the status of the door 12, the elapsed time since initiation of the decontamination cycle, and the state of the sensor 58.

At the end of the predetermined decontamination cycle time, the controller 40 removes power from the lamps 16, 18 and 20 and waits about twenty minutes before advising the user that it is safe to open the mailbox 4 by so indicating on the externally visible indicators 50 and 52. The additional twenty minute wait time ensures that substantially all of the ozone produced during the decontamination cycle has been converted back to oxygen.

It is assumed that the next opening of the door 12 is for the purpose accessing and removing the contents, i.e., the mail from within the mailbox 4. After the door 12 is closed, as determined by the controller 40 in response to the state of the switches 44 and 46, and assuming the sensor 58 indicates an “empty” state, the contamination cycle events set forth above are repeated for a duration of about 50% of the contamination interval to ensure that all internal surfaces of the mailbox 4 have been decontaminated. The mailbox 4 is then in condition to accept mail, after which the decontamination cycle as set forth above is repeated.

If at any time during the decontamination or cleaning cycles (that is, whenever the lamps 16, 18 and 20 are operative), and the door 12 is opened, the controller 40 de-energizes the lamps 16, 18 and 20. As an additional protective mechanism, the controller 40 operative in conjunction with the sensor 30 is calibrated to a dark level by performing the calibration process with the door 12 closed. If at anytime during operation of the lamps 16, 18 and 20 the door is opened or an opening is made in the mailbox 4 (such as for example, by a bullet), the controller recognizes an out-of-calibration situation and de-energizes the lamps 16, 18 and 20.

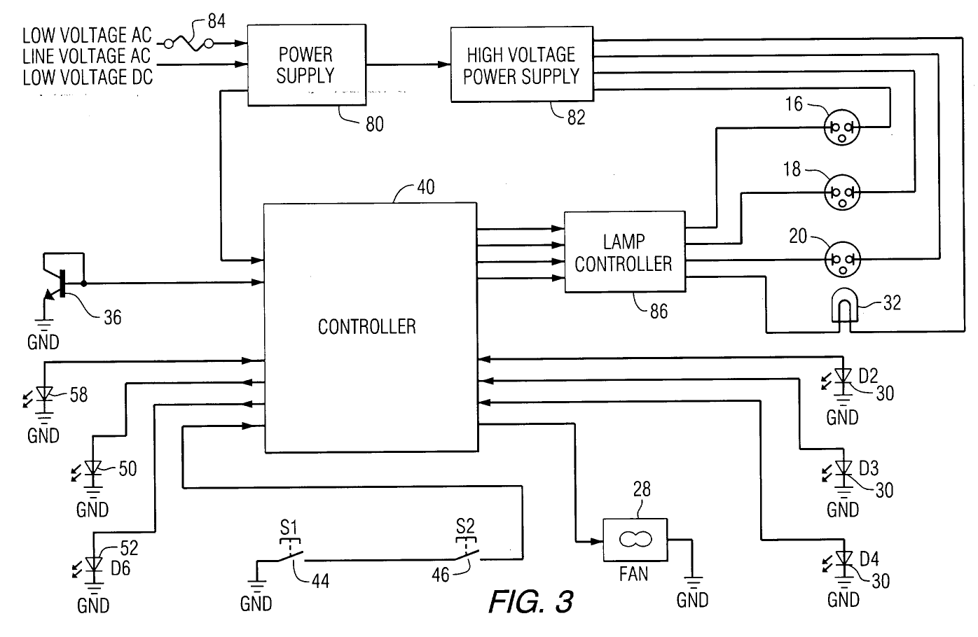

FIG. 3 is a schematic diagram depicting the various elements of the mailbox 4 controlled and/or monitored by the controller 40, which as described above, provides monitoring functions, conditions the sensor inputs and in response to these inputs controls the elements of the mailbox 4.

A power supply 80 provides power for the controller 40 and certain of the various electrical components of the mailbox 4, including supplying power to an ultra-violet lamp high-voltage power supply 82. In one embodiment, the high-voltage power supply 82 comprises a transformer to boost the power supply voltage to the higher value required to energize the ultra-violet lamps 16, 18 and 20. Several options are illustrated for supplying external power to the power supply 80, including standard 120 VAC via a fuse 84, 12 or 24 VAC supplied from a step-down transformer (not shown) via the fuse 84, and DC voltage supplied from an uninterruptible power supply (which in one embodiment requires conversion to AC for input to the high-voltage power supply 82).

The controller 40 performs the necessary monitoring and control functions according to logic and sensing elements. Alternatively, the controller comprises a micro-controller executing software programs for performing the necessary monitoring and control functions.

A lamp controller 86 is responsive to the controller 40 for controlling the lamps 16, 18 and 20. As described above, in the preferred embodiment, the lamp controller 86, in response to signals from the controller 40, energizes the lamps 16, 18 and 20 when the sensor 58 indicates that material is present in the mailbox 4 and when both of the switches 44 and 46 are in a condition indicating that the door 12 is closed.

Preparatory to energizing the lamps 16, 18 and 20 it may be necessary to raise the internal temperature of the mailbox 4 for more effective UV-C production from the lamps 16, 18 and 20. This is accomplished by a signal from the controller 40 to the heat lamp 32. When the internal temperature has reached a predetermined value, as measured by the temperature sensor 36 and input to the controller 40, the lamps 16, 18 and 20 are energized. As discussed above, the UV radiation produced by the lamps 16, 18 and 20 collaterally produces ozone that aids in the decontamination of the contents of the material within the mailbox 4 and also in the space between individual material items that are not exposed to the ultra-violet radiation.

The fan 28 is controlled by the controller 40 to raise the internal pressure of the mailbox 4 and further to prevent the formation of temperature gradients within the mailbox 4 that can reduce the effects of the UV-C radiation. The internal pressure also provides improved penetration of the ozone into and between material (e.g., letters) within the mailbox 4.

The lamps 50 and 52 are energized by the controller 40 to indicate that the mailbox 4 can be safely opened, or that the unit is executing the decontamination process and should not be opened.

Although not illustrated in the cutaway view of FIG. 1, the power supply 80, the high-voltage power supply 82, the lamp controller 86 and the controller 40 can be collocated and disposed within the chamber 14 in any one of several locations so as not to interfere with the placement of mail and parcels within the chamber 14. In another embodiment these components can be separately dispersed within the chamber. For example, the high-voltage power supply 82 and the lamp controller 86 can be positioned proximate the lamps 16, 18 and 20.

An apparatus and process have been described as useful for forming a decontaminating mailbox for killing various pathogens that may be on or within mail placed within the mailbox. While specific applications and examples of the invention have been illustrated and discussed, the principals disclosed herein provide a basis for practicing the invention in a variety of ways and in a variety of situations. Numerous variations are possible within the scope of the invention. The invention is limited only by the claims that follow.

What is claimed is:

1. An enclosure for decontaminating an item therein, comprising:
an ultra-violet energy source disposed within the enclosure;
an ozone-producing source disposed within the enclosure; and
an air circulator for circulating air within the enclosure.

2. The enclosure of claim 2 wherein the item comprises an outer enclosure surrounding an inner content element, wherein the ultra-violet energy has the effect of destroying pathogens on the surface of the item and the ozone has the effect of destroying pathogens within the item through the penetration of the outer enclosure.

3. The enclosure of claim 1 comprising a mailbox, and wherein the item comprises mail.

4. The enclosure of claim 1 wherein the ultra-violet energy source comprises one or more ultra-violet energy producing lamps.

5. The enclosure of claim 1 wherein the ultra-violet energy source produces ultra-violet energy in the UV-C ultraviolet band.

6. The enclosure of claim 1 further comprising a heat source for increasing the temperature of the enclosure to a predetermined temperature at which temperature the ultra-violet energy produced by the ultra-violet energy source is optimal.

7. The enclosure of claim 1 further comprising an ultra-violet sensor for determining the ultra-violet energy produced by the ultra-violet energy source, wherein the ultra-violet energy source is controlled in response to the determined ultra-violet energy.

8. The enclosure of claim 1 wherein the ultra-violet energy source produces ultra-violet energy having a wavelength of between about 100 and 280 nanometers.

9. The enclosure of claim 1 further comprising an access opening providing access to the interior of the enclosure such that items can be placed within and removed from the enclosure, and wherein a door covers the access opening.

10. The enclosure of claim 9 further comprising one or more door switches for determining the position of the door relative to the access opening.

11. The enclosure of claim 10 wherein the ultra-violet energy source is controlled in response to the door position.

12. The enclosure of claim 10 wherein the one or more door switches comprise a first and a second door switch, and wherein the first and the second door switches are electrically connected in series, and wherein the ultra-violet energy source is operative only when both the first and the second door switches indicate the door is in a closed position.

13. The enclosure of claim 1 further comprising a sensor for determining the presence of items within the enclosure and for controlling the ultra-violet energy source in response thereto.

14. The enclosure of claim 13 wherein the sensor is selected from among a mechanical switch having force-driven contacts responsive to the weight of the item for changing the position of the switch contacts, and a light-sensing sensor.

15. The enclosure of claim 1 wherein a ultra-violet lamp operating in the UV-C range of ultra-violet frequencies produces collateral ozone.

16. The enclosure of claim 1 wherein the air circulator is selected from among a fan and an air blower.

17. A mailbox for decontaminating a parcel of mail, comprising an envelope enclosing a contents item, placed therein, comprising:
an ultra-violet lamp disposed within the enclosure, and wherein the ultra-violet lamp produces ozone; and
an air circulator for circulating air within the enclosure so as to penetrate the envelope and decontaminate the contents item.

18. A method for decontaminating a porous item comprising an outer enclosure and contents, comprising:
exposing the item to ultra-violet energy;
causing tri-atomic (O3) oxygen to penetrate into the porous item to decontaminate the contents.
